(19) United States
(12) Patent Application Publication
(10) Pub. No.: US 2008/0220442 A1
(43) Pub. Date: Sep. 11, 2008

(54) Difference detection methods using isoelectric focusing chips

(75) Inventor: Jian Liao, Union City, CA (US)

Correspondence Address: BEYER WEAVER LLP, P.O. BOX 70250, OAKLAND, CA, 94612-0250, US

(73) Assignee: Proteinics

(21) Appl. No.: 11/998,166

(22) Filed: Nov. 27, 2007

Related U.S. Application Data
(60) Provisional application No. 60/868,865, filed on Dec. 6, 2006.

(51) Int. Cl. G01N 33/53

(52) U.S. Cl. 435/7.1; 204/403.01

(57) ABSTRACT

The present invention provides an isoelectric focusing difference detection method, as well as an isoelectric focusing chip and device. The method entails labeling proteins in the first sample with a first label, and labeling proteins in the second sample with a second, different label, wherein each label has ionic and pH characteristics whereby, upon isoelectric focusing, relative migration of a protein labeled with the first label is substantially the same as relative migration of the same protein labeled with the second label. The samples are subjected to isoelectric focusing to produce a pattern of separated proteins from both samples, which is then detected to compare the protein composition of the samples. The method can be carried with a novel isoelectric focusing chip including a substrate comprising a channel for carrying out isolectric focusing. The substrate includes a non-fluorescent or low-fluorescence material in a detection region of the channel, facilitating direct detection of the pattern of separated proteins, which makes the chip particularly useful for clinical applications, including use disease diagnosis and monitoring.

### CROSS-REFERENCE TO RELATED APPLICATIONS

This application claims the benefit of U.S. Provisional Application No. 60/868,865, filed Dec. 6, 2006, which is hereby incorporated by reference in its entirety.

### FIELD OF THE INVENTION

The present invention relates to generally to the area of isoelectric focusing methods, chips, and devices for detecting differences in protein composition of samples. In particular, the invention relates to methods, chips, and devices useful in diagnosing disease, monitoring disease progress, and/or discovering biomarkers.

### BACKGROUND OF THE INVENTION

Researchers studying the functional differences between cells use a variety of tools to detect and monitor differences in cell differentiation, structure, function and development. The greatest insight into functional differences can be obtained by studying the differences and similarities in the protein composition and protein modification (including phosphorylation, glycosylation and protelytic digestion) between the different cell types, cell stages, cells from different points in disease progression, and cells affected by different disease conditions. Methods and devices useful in determining the differences in the protein content and modification in samples from, e.g., normal and diseased tissues, healthy and ill subjects (with and without drug treatment); wild-type and mutant cells, can provide valuable information leading to important diagnostic or therapeutic applications, as well as valuable tools in clinic diagnosis.

Biological samples containing mixtures of proteins and/or peptides can be separated into individual components by various means, including mass spectrometry, electrophoresis, and chromatography. Separation according to differences in mass can be achieved by electrophoresing in a polyacrylamide gel under denaturing conditions. One dimensional and two dimensional gel electrophoresis have become standard tools for studying proteins. One dimensional Sodium Dodecyl Sulfate PolyAcrylamide Gel Electrophoresis (SDS-PAGE) through a cylindrical or slab gel reveals only the major proteins present in a sample tested. Two dimensional polyacrylamide gel electrophoresis (2D-PAGE), which separates proteins by isoelectric focusing (IEF), i.e., by charge in one dimension, and by size in the second dimension, is the more sensitive method of separation and will provide resolution of most of the proteins in a sample.

Proteins and peptides migrate in one- or two-dimensional gels as bands or spots, respectively. Especially small molecules including peptides can be separated by isoelectric focusing. In addition, the latter can resolve proteins or peptides, based on modifications, such as phosphorylation. The separated proteins and peptides are visualized by any of a variety of methods, such by staining with a protein-specific dye, by protein-mediated silver precipitation, autoradiographic detection of radioactively labeled proteins, and by covalent attachment of a label, such as a fluorescent molecule. Immediately following the electrophoresis, the resulting gel patterns may be visualized by eye, by fluorescent scanning, photographically, or by electronic image capture. Images can then be analyzed, for example, by computer-aided image analysis of digitized one or two dimensional gels.

In most conventional methods, IEF is run using tube IEF gels made by individual researchers, which have a high degree of variability, or IEF gels immobilized on a plastic supporting material. The plastic materials typically used to support IEF gels generally have the overlapping fluorescence absorbance with the conventional fluorescent labels, which interferes with direct scanning of gels when the separated proteins/peptides are fluorescently labeled.

Typically, comparisons of between protein samples have been made by comparing the results of running samples on different gels. The differences between the proteins, however, can be subtle. Imperfections in the gel can interfere with accurate observations. In order to minimize the imperfections, the gels provided in commercially available electrophoresis systems are prepared with exacting precision. Even with meticulous controls, two gels are never identical. IEF strips or gels may differ from one to the other in pH gradients or uniformity. In addition, the run-to-run electrophoresis conditions may also be different. Different software has been developed for automated alignment of different gels. However, most of the available software packages are based on linear expansion or contraction of one or both of the dimensions on two dimensional gels. The software cannot adjust for local distortions in the gels.

### SUMMARY OF THE INVENTION

The invention provides method of comparing the protein composition of at least two samples. The method entails labeling proteins in the first sample with a first label, and labeling proteins in the second sample with a second, different label, wherein each label has ionic and pH characteristics whereby, upon isoelectric focusing, relative migration of a protein labeled with the first label is substantially the same as relative migration of the same protein labeled with the second label. The samples are subjected to isoelectric focusing in a first pH gradient having a first pH range to produce a pattern of separated proteins from both samples. The pattern of separated proteins is detected prior to performing any additional separation step. In particular embodiments, no additional separation step is performed. If desired, the isoelectric focusing to produce a pattern of separated proteins from both samples can be carried out simultaneously in one or more additional pH gradient(s) having a different pH range from the first pH range. In particular embodiments, at least one additional pH gradient is adjacent, and parallel, to the first pH gradient, and the pattern of separated proteins in each gradient is detected by capturing a single image encompassing both patterns. In a variation of this embodiment, isoelectric focusing is performed in a plurality of pH gradients in parallel, wherein each pH gradient has a different pH range from every other pH gradient. The pH ranges can, collectively, define a continuous or discontinuous pH range. In particular embodiments, at least first and second samples are mixed after labeling to form a sample mixture, and the sample mixture is applied to the first pH gradient. The comparison of protein composition according to the invention can be quantitative.

The method of comparing protein composition can be carried out on biological samples. In exemplary embodiments, the protein composition of a test sample is compared to the protein composition of a reference sample. The comparison can provide information for diagnosis of one or more diseases or for monitoring the disease progression or treatment response.

The isoelectric focusing can be liquid or non-liquid isoelectric focusing. In particular embodiments, isoelectric focusing is carried out on an isoelectric focusing chip. In variations of such embodiments, the isoelectric focusing to produce a pattern of separated proteins from both samples can carried out simultaneously in one or more additional pH gradient(s) having a different pH range from the first pH range. For example, at least one additional pH gradient can be adjacent, and parallel, to the first pH gradient, and the pattern of separated proteins in each gradient can be detected by capturing a single image encompassing both patterns. In particular, isoelectric focusing can be performed in a plurality of pH gradients in parallel, wherein each pH gradient has a different pH range from every other pH gradient and. The pH ranges, can collectively, define a continuous or discontinuous pH range. In specific embodiments, the isoelectric focusing chip can be sealed or unsealed.

The proteins in the first and second samples can be labeled to saturation, though need not be. In particular embodiments, the first and second labels are luminescent labels, e.g., fluorescent labels, wherein each label emits luminescent light at a wavelength that is different from the wavelength at which luminescent light is emitted by any other label in the sample mixture. Where fluorescent labels are said isoelectric focusing is preferably carried out on an isoelectric focusing chip, wherein at least one surface of said chip comprises a non-fluorescent or low-fluorescence material.

The invention also provides an isoelectric focusing chip including a substrate comprising a channel for carrying out isolectric focusing. The channel includes first and second ends and a detection region between the first and second ends. Each end of the channel includes a receptacle for an electrode. The substrate includes a non-fluorescent or low-fluorescence material in the detection region of the channel. In particular embodiments, the channel is sealed along its length and additionally comprises a sample port at the first end of the channel. For example, the channel can be completely sealed, except for the openings for electrodes and the sample port, forming a chamber. In particular embodiments, the substrate includes one or more additional channels parallel to the first channel, each additional channel comprising first and second ends, and a detection region between the first and second ends, wherein each end of each additional channel comprises a receptacle for an electrode. In such embodiments, the substrate comprises a non-fluorescent or low-fluorescence material in the detection region of each additional channel. In exemplary embodiments, each channel is adjacent to at least one other channel, and the detection regions of each channel are aligned to permit detection by capture of a single image comprising all detection regions. In variations of such embodiments, the channels are sealed along their length, and each channel additionally comprises a sample port at one end of the channel. For example, each channel can be completely sealed, except for the openings for electrodes and the sample port, forming a plurality of chambers.

An isoelectric focusing chip according to the invention can include a gel in one or more chambers. The gel can include a pH gradient extending from the first end of the channel to the second end. In particular multi-channel embodiments, the pH gradient in each channel has a different pH range than that of the pH gradients of the other channels. The pH ranges can, collectively, define a continuous or discontinuous pH range.

Another aspect of the invention is an isoelectric focusing device including a support capable of holding an isolectric focusing chip in position; first and second electrodes for applying an electric field to an isoelectric focusing chip positioned on the support, wherein the support comprises a non-fluorescent or low-fluorescence material in a region underlying the detection region of the isoelectric focusing chip. The device can, optionally include a fluorescence detector attached to the support at a position opposite the detection region of an isoelectric focusing chip positioned on the support. In preferred embodiments, the isoelectric focusing device additionally includes means for controlling the temperature of the isoelectric focusing chip. The isoelectric focusing device can include, in particular embodiments, an isoelectric focusing control unit linked to the voltage source, whereby the isoelectric control unit controls flow of current to the first and second electrodes. If desired, the isoelectric focusing includes sample delivery means for introducing sample into the first end(s) of the channel(s). In particular embodiments, the isoelectric focusing device includes an isoelectric focusing control unit linked to the voltage source, whereby the isoelectric control unit controls flow of current to the first and second electrodes, and linked to the sample delivery means, whereby the isoelectric focusing control unit controls sample introduction. If the isoelectric focusing includes a fluorescence detector, the device can also include a detection control unit linked to the fluorescence detector, whereby the detection control unit operates the detector. In a variation of such embodiments, the isoelectric focusing control unit is additionally linked to the fluorescence detector, whereby the isoelectric focusing control unit operates the detector. The invention includes the isoelectric focusing device with or without an isoelectric focusing chip according to the invention positioned on the support.

### BRIEF DESCRIPTION OF THE DRAWINGS

FIG. 1 is a flow chart illustrating the isoelectric focusing difference detection method of the invention.

FIG. 2 illustrates a sealed isoelectric focusing chip according to the invention.

FIG. 3 illustrates an unsealed isoelectric focusing chip according to the invention

FIG. 4 illustrates a novel isoelectric focusing chip format, in which a plurality of different pH ranges, collectively, form a continuous pH range.

FIG. 5 illustrates a novel isoelectric focusing chip format, in which a plurality of different pH ranges, collectively, form a discontinuous pH range.

FIG. 6 illustrates a novel isoelectric focusing chip format, amenable to use in liquid or non-liquid isoelectric focusing, that provides a continuous pH range.

FIG. 7 illustrates an embodiment of the invention in which isoelectric focusing is carried out with multiple chips positioned vertically.

FIG. 8 illustrates novel chip holder that can be used to assemble individual chips into a discontinuous or continuous isoelectric focusing chip format. The chip holder can be used to hold the chips in position during isoelectric focusing and/or detection.

FIG. 9 illustrates an isoelectric focusing device for running isoelectric focusing vertically and detecting the results.

FIG. 10 illustrates the isoelectric focusing device of FIG. 9 with multiple isoelectric focusing chips in position for isoelectric focusing, followed by detection.

### DETAILED DESCRIPTION

The present invention provides difference detection methods wherein isoelectric focusing is carried out with two or more differently labeled samples, which permits intra-gel comparisons of different protein samples or of a particular sample with an internal reference, such as a control. The methods can be carried out in any of a number of novel formats in which the pattern produced by side-by-side isoelectric focusing separations on different pH gradients, having different pH ranges, can be captured in a single image per label. Image analysis can then be carried out to identify qualitative and quantitative differences between the samples. This approach can be employed clinically to diagnose or provide information about disease states, in addition to having utility in other commercial or research applications. The invention also provides chips and devices adapted for performing isoelectric focusing, followed by detection in situ, to facilitate clinical as well as other commercial or research use of the methods of the invention. The chips are adapted for direct detection in that they include non-fluorescent or low-fluorescent materials, at least on the detection side of the chip.

### DEFINITIONS

Terms used in the claims and specification are defined as set forth below unless otherwise specified.

The terms “protein” and/or “polypeptide” are used herein to refer a polymer of amino acids, and unless otherwise limited, include atypical amino acids that can function in a similar manner to naturally occurring amino acids. Proteins/polypeptides include full-length molecules, as well as fragments, and modified isoforms.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N.Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

As used with reference to a protein or polypeptide, the term “full-length” refers to a polypeptide having the same length as the mature wild-type polypeptide.

The term “fragment” is used herein with reference to a polypeptide or to describe a portion of a larger molecule. Thus, a polypeptide fragment can lack an N-terminal portion of the larger molecule, a C-terminal portion, or both. Polypeptide fragments are also referred to herein as “peptides.”

The term “modified isoform” is used herein with reference to full-length and fragment polypeptides that are post-translationally modified either in vivo or in vitro. Examples of modified isoforms include polypeptides modified by phosphorylation, glycosylation, or other moieties, such as polyethylene glycol.

The term “relative migration” is used herein to refer to the migration of a protein during isoelectric focusing relative to another protein, such as a standard. If two proteins have substantially the same relative migration, after isoelectric focusing, these proteins are detected at the substantially the same point in a pH gradient.

In a plurality of pH gradients, the pH range of each gradient can, collectively, define a “continuous pH range,” for example: pH 3.0-3.5, 3.5-4.0, 4.0-4.5, 4.5-5.0, 5.0-5.5, etc. In a continuous pH range, the endpoint of a lower range will be the starting point of the next range. In a continuous pH range, therefore, all pHs between the lowest pH and the highest pH are represented.

Alternatively, a plurality of pH gradients can, collectively define a “discontinuous pH range,” for example: pH 3.0-3.5; 4.0-4.5, 5.0-5.5. Thus, a discontinuous pH range can have gaps, where specific pHs are not represented. Another example of a discontinuous pH range is: pH 4.0-4.5, 3.0-4.0, 5.0-5.5, 4.5-5.0. In this example, the all pHs are represented, but are not in order. These formats are used for specific types of disease diagnosis and monitoring.

In any given plurality of pH gradients, the pH ranges for each individual gradient can represent the same increment in pH as for any other gradient or can be different.

The term “channel” refers to an elongate path in a substrate. The term “channel” encompasses a channel that is open along its length, a channel that is formed as an open channel and then sealed, e.g., by placement of a cover over the channel. A “sealed channel” also includes, for example, a chamber. In an exemplary chamber, the substrate is a tube.

As used herein, the term “protein composition,” unless otherwise indicated, refers to the type and amount of proteins and peptides present in a sample. Different types of proteins detectable using the methods described herein include proteins having different amino acid sequences, as well as differently modified or degraded versions of the same proteins.

### I. Difference Detection Methods

In General

The invention includes a method of comparing the protein composition of at least two samples. The method employs a matched set of labels wherein each label in the set is generally equal to the other labels in ionic and pH characteristics, and chemical reactivity for covalent attachment to proteins, yet is detectably different. Most conveniently, sets of fluorescent labels are employed, wherein each label fluoresces at a different wavelength, thereby exhibiting a different color luminescence when viewed. The labels can be roughly equal in molecular weight, but need not be. Each one of the labels within the matched set of labels is used to label proteins in a different sample to be compared. After labeling, the samples are subjected to isoelectric focusing in a pH gradient to produce a pattern of separated proteins. The relative migration of a protein labeled with a first label in a set is substantially the same as the relative migration of a protein labeled with a second label in a set. Accordingly, a qualitative difference between the protein samples is detected as a signal from one label, but not another at a given point in the pattern of separated proteins. A quantitative difference between the samples is detected by comparing the relative intensities of the signals from each label at a given point in the pattern (e.g., percentage of labeling in each band). Signal is detected prior to any additional separation step, such an electrophoretic separation based on molecular weight (as in 2-dimensional electrophoresis). In particular embodiments, no additional separation is preformed.

The method can be carried out by performing isoelectric focusing in one or more additional pH gradient(s) having a different pH range from the first pH range. In particular embodiments, the additional pH gradient(s) is/are adjacent, and parallel, to the first pH gradient, such that the pattern of separated proteins in each gradient can be detected by capturing a single image encompassing each pattern. For example, isoelectric focusing can be performed in a plurality of pH gradients in parallel, wherein each pH gradient has a different pH range from every other pH gradient. The pH ranges of the plurality of gradients can, collectively, define a continuous pH range or a discontinuous pH range.

Samples

The samples can be derived from any two sources, the protein content of which one wishes to compare or contrast. For example, a first sample can be derived from (e.g., a cell extract) wild-type, or normal, cells, and a second sample can be derived from mutant cells (e.g., transformed cells) or a disease sample from the same species. Alternatively, a first sample can be derived from normal cells and a second sample can be derived from cancerous cells from the same individual. Cells from the same individual at different stages of development or different phases of the cell cycle can be compared as well. The differences in protein composition between cells of the same type from different species can also be analyzed by the methods described herein. In addition, these methods can be used to monitor how cells respond to any of variety of stimuli or drugs. Any event that alters cell behavior as expressed through protein changes can be detected without the need and expense of complicated high precision 2D-PAGE systems.

In particular embodiments, the protein composition of a test sample is compared to the protein composition of a reference sample. The reference sample is known to have a feature of interest, e.g., it is derived from normal cells, and the comparison is carried out to determine whether the test sample differs from the reference sample with respect to the presence or amount of one or more proteins. The significance of any differences may not be known when the analysis is carried, as, for example, in the process of identifying disease markers. Any difference found in comparing a disease sample to a normal sample represents a putative disease marker, which can then be validated in the usual way. Alternatively, if one or more disease marker(s) is/are known, a putative disease sample may be compared with a normal sample to provide diagnostic, prognostic, or disease progression information.

“Biological samples” that can be analyzed using the methods of the present invention include biological fluids, such as whole blood, serum, plasma, synovial fluid, cerebrospinal fluid, bronchial lavage, ascites fluid, bone marrow aspirate, pleural effusion, urine, as well as tumor tissue or any other bodily constituent or any tissue culture supernatant.

Samples are obtained and prepared for isoelectric focusing according to standard methods. If the sample includes intact cells or tissues, proteins can be extracted from the cells or tissues using cell lysis buffer. The sample can be pretreated as necessary by dilution in an appropriate buffer solution or concentrated, if desired. Any of a number of standard aqueous buffer solutions, employing any of a variety of buffers, such as phosphate, borate, carbonate, or the like, e.g., at physiological pH, or higher pH, can be used and followed-up by buffer exchange for protein labeling and isoelectric focusing.

Labeling of Samples

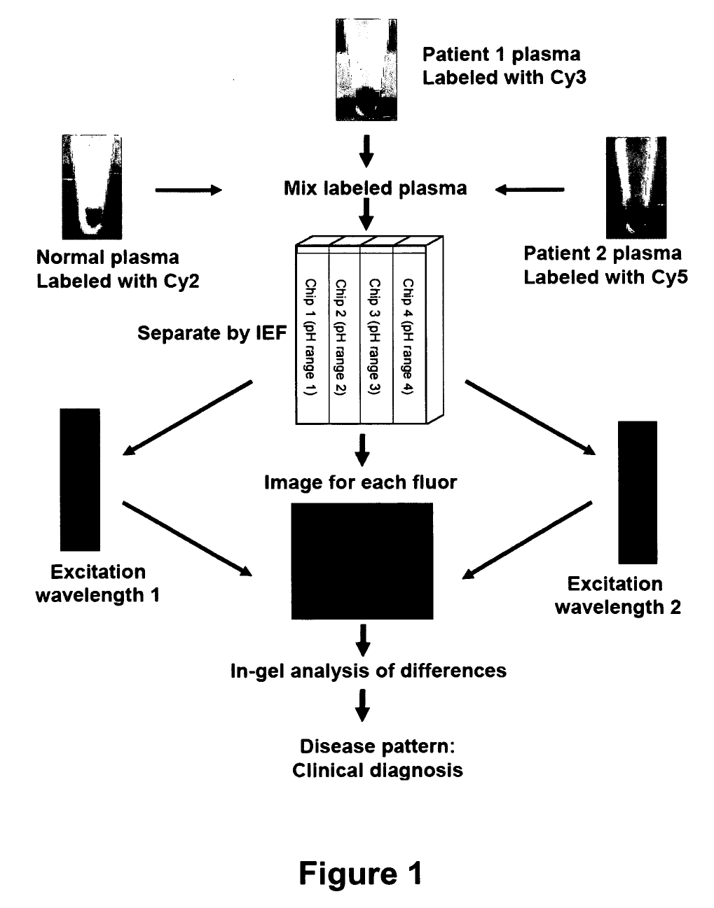

With reference to the schematic diagram of FIG. 1, a first sample is labeled with the first label of a matched set of labels. A second sample is labeled with the second label of the matched set. A normal sample is labeled with the third label of the matched set. To label a sample, a reactive form of the label and the sample are incubated for a period of time sufficient to allow the formation of a covalent bond between the reactive form of the label and potential attachment or binding sites on the proteins in the sample. The period of time can be, e.g., from about 15 to about 60 minutes, depending on the labeling materials used. The temperature range is generally from about 0° C. to about 4° C. The reaction between the label and the proteins may be quenched after a sufficient percentage of available binding sites on the protein molecules are covalently bound to the label. Any suitable known quenching material may be used. The labeling can be quenched prior to saturation of the available binding sites or can be carried out to saturation. Because the method does not require a separation based on molecular weight, any change in molecular weight due to labeling does not affect the analysis. Any matched-charge change does not affect the analysis or even help the analysis for the proteins with extreme pI (very low or very high pI).

The label can be any material having a detectable physical or chemical property. In particular embodiments, the labels employed are luminescent labels, wherein each label emits luminescent light at a wavelength that is different from the wavelength at which luminescent light is emitted by any other label in the sample mixture.

Selection and synthesis of a matched set of labels is known and described, for example, in U.S. Pat. No. 6,127,134 (issued Oct. 3, 2000 to Minden et al., which is incorporated herein by reference). Briefly, labels are covalently coupled to proteins, preferably via lysine residues of the proteins, but coupling may also be to sulfhydryl or carboxylic acid groups in the proteins. Regulation of the pH of proteins to force attachment of labels at one amino acid residue to the exclusion of other amino acids is a well known technique, as set forth in R. Baker, Organic Chemistry of Biological Components, (Prentice Hall, pub. 1971). For analysis of proteins, a plurality of attachment sites are labeled. The optimum percentage of attachment sites labeled will depend on the labels chosen. In certain embodiments, when using the exemplary labels discussed below, preferably no more than 2% of the attachment sites and more preferably, slightly less than 1%, are labeled, to avoid rendering the protein insoluble. When lysine is the attachment site, the covalent linkage destroys the positive charge of the primary amine of the lysine. Because isoelectric focusing depends on charge, it is important to compensate for the charge loss. A basic residue should remain basic. Changing the pKₐ of one residue per protein by as much as 3 can be tolerated, provided the basicity or acidity of the modified residue, as the case may be, is not altered. Labels like rhodamine and fluorescein are not suitable for the labeling to saturation because of the difference in charge. In this case, minimal labeling (1-3% of total protein labeled, and only one labeling per molecule) can give good results as long as the changed charges are matched in the first and other labelings.

In certain embodiments, labeling to saturation at cysteine residues, for example, may provide better results than labeling at more frequently occurring residues such as lysine. More specifically, saturation cysteine labeling can improve detection after isoelectric focusing by reducing or avoiding quenching that can occur due to a large amount of lysine labeling in the same molecules. Saturation labeling via protein peptide end-labeling (either N-terminus or C-terminus) can also provide results regardless of the size of the labeling materials as long as the labels are matched for any change in charge.

The invention also encompasses methods in which the protein charge is changed before, during, or after labeling to facilitate isoelectric focusing. For example, labeling resulting in a negative charge change (e.g., employing sulfonate) can prevent the quench of the fluorescent signal. In addition, changing the charge after labeling can be advantageous for isoelectric focusing detection of high pI proteins and/or low pI proteins. In conventional isoelectric focusing, it is difficult to separate high pI proteins and/or peptides because the reducing reagents normally used have pIs around pH5.0 (e.g., dithiothreitol). However, according to some embodiments of the invention, labeling can be carried out so as to shift the protein pI shift down 4 pH units, e.g., from pH9-12 to pH5-8 to enhance separation for proteins with pIs between pH9-12. Conversely, if desired, labeling can carried out to shift protein pI up 3 pH units from pH2-5 to pH5-8 to give much better resolution for proteins with pI between pH 2-5. Methods in which the protein charge is changed to facilitate separation are equally applicable to 2-D electrophoresis.

Suitable labels include the fluorescent cyanine labels described in Mujumdar, R. B. et al., “Cyanine label labeling reagents containing isothiocyanate groups,” Cytometry 10:11-19 (1989) and Waggoner et al., U.S. Pat. No. 5,268,486 entitled “Method for labeling and detecting materials employing arylsulfonate cyanine labels” issued in 1993 and incorporated herein by reference. The cyanine labels have the following general structure:

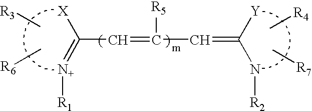

where X and Y can be O, S or (CH₃)₂—C, m is an integer from 1 to 3 and at least one of R₁, R₂, R₃, R₄, R₅, R₆ or R₇ is a reactive group which reacts with amino, hydroxy or sulfhydryl nucleophiles. The dotted lines represent the carbon atoms necessary for the formation of one ring to three fused rings having 5 to 6 atoms in each ring. R₃, R₄, R₆ and R₇ are attached to the rings. The reactive moiety can be any known reactive group. Reactive groups that may be attached directly or indirectly to the chromophore to form R₁, R₂, R₃, R₄, R₅, R₆ or R₇ groups may include reactive moieties such as groups containing isothiocyanate, isocyanate, monochlorotriazine, dichlorotriazine, mono- or di-halogen substituted pyridine, mono- or di-halogen substituted diazine, maleimide, aziridine, sulfonyl halide, acid halide, hydroxysuccinimide ester, hydroxysulfosuccinimide ester, imido ester, hydrazine, azidonitrophenyl, azide, 3-(2-pyridyl dithio)-proprionamide, glyoxal, and aldehyde.

The cyanine labels described in the Waggoner et al. patent attach to the protein via the activated ester of hexanoic acid. While the coupling destroys the charge of the lysine side chain, the intrinsic charge in the label compensates. It in effect moves the charge away from the protein molecule but maintains the same overall charge within the sample to be electrophoresed. In the cyanine label molecule, two functionalized indole rings are connected via a polyene linker. The spectral characteristics of cyanine labels can be easily modulated by simply changing the length of the linker between the indole rings of the label. A longer or shorter linker length will result in fluorescence at different wavelengths and thus, different colors. Changing the length of the linker changes the molecular mass of the label and thus the molecular weight of the labeled protein. In particular embodiments of the invention, detection is carried out after isoelectric focusing, and no separation based on molecular weight is performed. Therefore, the analysis is not affected by molecular weight changes due to labeling. In addition, removing or adding charges to the labeling molecules are tolerated in the minimal labeling and protein/peptide end-labeling, and these even give improved separation of the protein peptides with extreme pIs (>pH10 or <pH5), as long as the labels employed produce similar changes in protein/peptide charge.

Other exemplary labels useful in the invention have the general formula:

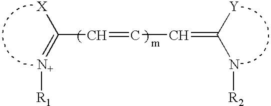

wherein X and Y equal S, O, or CH₃—C—CH₃, m is an integer from 1 to 3 and either R₁ or R₂ is a reactive group capable of covalently binding to protein, such as the reactive groups described above for the unmodified cyanine labels. The dotted lines represent 1, 2 or 3 fused rings having 5 or 6 carbon atoms in each ring. Each side should balance the other side.

An example of a matched pair of labels developed according to the general formula follows:

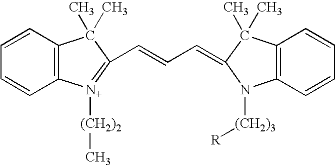

(Propyl Cy-3-NHS) which fluoresces red and,

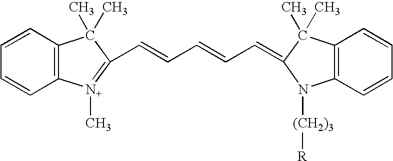

(Methyl Cy-5-NHS) which fluoresces far red in the spectrum wherein R is a reactive group. As stated above, O or S or a combination thereof can be placed in the X and Y positions in place of (CH₃)₂ C—.

The cyanine labels are one choice for the matched set of labels of the present invention. Other label compounds may be used in place of the cyanines, such as dipyrromethene boron difluoride labels, the derivatized 4,4-difluoro-4-bora-3a,4a,-diaza-S-indacene labels, described in U.S. Pat. No. 4,774,339 to Haugland et al. and incorporated herein by reference, which are sold by Molecular Probes, Inc. under the trademark BODIPY®. The BODIPY® labels, which have no net charge, are covalently linked to lysine side chains using an activated n-hydroxysuccinimidyl ester which forms an amide bond. The result is the loss of the lysine positive charge. Therefore, a positively charged linker group is used in the matched labels of the invention to replace the lost primary amine with the linker's tertiary amine. The procedures for making BODIPY® labels are described in U.S. Pat. No. 4,774,339. Addition of the positively charged linker can be carried out by any technique well known to those skilled in the art. A linker can be designed with three functional groups; (1) to react with the BODIPY®-NHS ester, (2) to carry the desired charge, and (3) to be activated so that the BODIPY®-linker construct will react with specific amino acid residues of the proteins in the sample.

The major considerations for the matched set of labels are the maintenance of charge and distinct and different spectral characteristics. Any neutral labels with a positive linker or any positively charged labels, preferably each having a +1 charge, that otherwise satisfy the requirements described herein can serve as the labels in the matched set of labels of the present invention. The intrinsic positive charge of cyanine labels can be advantageously used to replace the positive charge of lysine. The pKₐ of cyanines and lysine are rather different; however, conditions can be selected for label:protein ratio to be less than one. This low level of labeling ensures that there will be negligible changes in the protein's migration on isoelectric focusing. Labels may be used which match the pKₐ of lysine more closely. Alternately, labels that modify other amino acid residues may be used, provided the amino acid's ionic characteristics are substantially preserved by the modification. Instead of a lysine, the attachment site on the protein may, for example, be a sulfhydryl or carboxylic group. When a sulfhydryl group is the attachment site on the protein, the corresponding attachment site on the label can be an iodoalkyl group. When a carboxylic acid group is the attachment site on the protein, the corresponding attachment site on the label can be a chloroketone or a carbodiimide. In certain embodiments, minimal labeling (e.g., 1-3%, using labels that either change or do not affect protein/peptide charge), followed by affinity purification (for example, where the labeling dyes carry one or more ligand recognized by a binding partner, such as an antibody, 6×His-tag, avidin or biotin) before loading onto isoelectric focusing chips can improve resolution because the samples are cleaner and more concentrated.

Isoelectric Focusing

The labeled samples can mixed and, as illustrated in FIG. 1, applied in measured aliquots to one or more different pH gradients and subjected to isoelectric focusing.

Isoelectric focusing (often called electrofocusing) is a method for separating molecules based on differing charge characteristics. For isoelectric focusing of proteins, the protein mixture is subjected to an electric field in an inert medium in which a stable pH gradient has previously been generated. The anode region is at a lower pH than the cathode, and the pH range is chosen such that the proteins to be separated have their isoelectric points within this range. A protein which is in a pH region below its pI will be positively charged and so will migrate towards the cathode. However, as it migrates, so the pH will decrease until the protein reaches a pH which is its pI. At this point it has no net charge and so migration ceases.

The stable pH gradient between the electrodes is formed by including a mixture of low molecular weight “carrier ampholytes” in the inert medium. Suitable carrier ampholytes include synthetic, aliphatic polyaminopolycarboxylic acids available commercially whose individual pI values cover a preselected pH range. Carrier ampholytes spanning a wide pH range (e.g., pH 3-10) or a narrow range (e.g., pH 7-8) can be purchased commercially from, e.g., Ampholine, BioLyte, and Pharmalyte.

The exact format for isoelectric focusing depends on the nature of the inert medium used. Isoelectric focusing can be carried out in liquid or non-liquid formats. Early applications of isoelectric focusing used water-cooled vertical glass columns filled with carrier ampholytes in a sucrose density gradient. The sucrose density gradient was the inert medium, the density gradient helping to stabilize the liquid column against convective mixing due to heat generated during electrophoresis. The upper electrode (anode) was connected to a reservoir of acid (e.g., phosphoric acid) and the lower electrode to a reservoir containing an alkaline solution (e.g., NaOH). After a period of electrophoresis to establish the pH gradient, the protein sample was introduced into the column and electrophoresis continued until the proteins had reached their pI's. The individual protein bands could then recovered by draining the column from its base into a series of fractions. This format is useful for preparative applications.

The methods of the invention can be used in conjunction with preparative applications, but it is anticipated that the methods of the invention be used primarily in analytical applications, and diagnostic applications, in particular. Analytical isoelectric focusing can be carried out either in vertical polyacrylamide rod gels or in horizontal polyacrylamide or agarose slab gels, for example. In particular embodiments, isoelectric focusing is carried out on an isoelectric focusing chip, such as those described below.

Detection

Proteins that the two samples have in common form coincident bands upon isoelectric focusing. In the case of fluorescent labels, the ratio of the fluorescence intensity of signal from identical proteins in each sample will be constant for the vast majority of proteins. Proteins that the two samples do not have in common will migrate independently. Thus, a protein that is unique or present in a different concentration in one sample relative to the other will have a different ratio of fluorescence intensity from the majority of protein spots. For example, the proteins that are in the first sample may be labeled red, while the second group is labeled blue. Under conditions where exactly equal amounts of protein from each group is mixed together and run on the same gel the ratio of fluorescence intensity could be one for the majority of proteins (the actual ratio corresponding to substantially equivalent amounts of the same protein may deviate from one, depending on the labels used and the detection conditions). Those proteins that are present in different amounts in each sample will have a fluorescence intensity ratio less than or greater than one (or greater or lesser than the ratio detected for substantially equivalent amounts of the same protein). In the example shown in FIG. 1, the fluorescence intensity ratios can be calculated for any desired comparison. For example, a ratio can be calculated to compare the two patient samples with one another or ratios can be calculated to compare each patient sample to the normal sample.

For fluorescently labeled proteins, the pattern of separated proteins in the isoelectric focusing chip can be analyzed, for example, by fluorescence scanner that detects two or more different wavelengths, by a fluorescent microscope or by any known means for detecting fluorescence. Gel analysis can be completely automated by means of computer-aided identification of protein differences. Using an electronic detection system such as a laser scanning system with a photo multiplier tube or a charged-coupled device (CCD) camera and a white light source, two or more electronic images are made of the separated proteins using different known filter sets to accommodate the different spectral characteristics of the labels. Each image captures fluorescence of a given label using a filter that filters out all light except that emitted at the wavelength of the label. Exposure can, for example, be in the range of about 5 to about 500 seconds. The differences in the samples can be identified, either during electrophoresis or preferably in less than ½ hour following electrophoresis. Several software packages are commercially available which will either subtract the first image from the second to identify spots/bands that are different, or, alternatively, the images may be divided to leave only the spots/bands not common to both images. In subtracting the images, like spots will cancel each other, leaving only those that are different. In ratio analysis, like spots can be normalized to a value of one. Differences will result in values greater than one less than one.

In particular embodiments, where isoelectric focusing is performed in a plurality of pH gradients in parallel, the pattern of resulting separated proteins is analogous to spots on a nucleic acid array. Nucleic acid array hybridizations typically employ fluorescent labels and routinely employ multiple fluorescent labels. Accordingly, detection systems developed for these applications can be readily adapted to use in the isoelectric focusing methods and devices of the invention. See, e.g., U.S. Pat. Nos. 6,562,565 and 6,465,182 (which are both incorporated by reference herein).

As those of skill will readily appreciate, the principles described herein can be adapted to the analysis of nucleic acids. For example, nucleic acids can be end-labeled or minimally labeled (about 1-3%) with molecular weight-matched fluorescent dyes and then separated on an agarose gel. In exemplary health monitoring applications, DNA samples collected from a subject at different ages can be digested with restriction enzymes and then labeled with different fluorescent dyes, as described above, and separated, for example, on a conventional DNA sequencing gel, followed by scanning and image analysis to provide an early indication of disease.

### II. Isoelectric Focusing Chip

The invention also includes an isoelectric focusing chip. The chip includes a substrate that supports the inert medium (e.g., a gel) in which the pH gradient(s) is/are generated. The chip can be provided without the inert medium or with the inert medium, with or without ampholytes for generating a pH gradient. The pH gradient can be preformed or can form upon application of an electric field. In preferred embodiments, the chip is ready to use, with a preformed pH gradient. Conveniently, the substrate includes a channel having a receptacle for an electrode at each end and a detection region between the two ends of the channel. The substrate preferably has a non-fluorescent or low-fluorescence material in the detection region of the channel to facilitate fluorescence detection following isoelectric focusing, without the need to transfer the isoelectric focusing medium to a detector.

The basic considerations for design and preparation of isoelectric focusing chips is well known as horizontal slab gel isoelectric focusing is now the most widely used format for electrofocusing. Thin sheets of polyacrylamide or agarose gel containing carrier ampholytes chosen to give the correct pH range are mounted on plates can be purchased or prepared in the laboratory. It is important to avoid molecular sieving effects so that the protein separation occurs solely on the basis of charge. Hence agarose gels are typically chosen in preference to polyacrylamide gels for the separation of larger proteins because of their larger pore size. If polyacrylamide gel is used, the gel concentration is chosen so as to give a large pore gel and so minimize molecular sieving. Gel thicknesses of 1-2 mm can be used but increasingly ultra-thin gels only 0.1-0.25 mm thick are being used since these are cheaper (carrier ampholytes are expensive) and allow shorter running times (since higher voltages can be used) and improved resolution.

Any suitable substrate can be employed in the isoelectric focusing chip. As noted above, the substrate preferably includes a non-fluorescent or low-fluorescence material in the detection region of the channel. Such materials are well known and include, for example, glass, quartz, or plastic. Plastics suitable for use as array substrates include any that produce about 200 percent or less of the fluorescence compared to glass of 100-100 microns thickness at excitation wavelengths between about 290, 300, 310, 320, or 350 to about 300, 340, 370, 400, or 420 nm and at emission wavelengths between about 290, 300, 350, 400, or 500 and about 400, 600, 800, or 1000 nm. An exemplary low-fluorescence plastic is cycloolefin, and examples of non-fluorescent materials include clear polyethylene, silicon, etc.

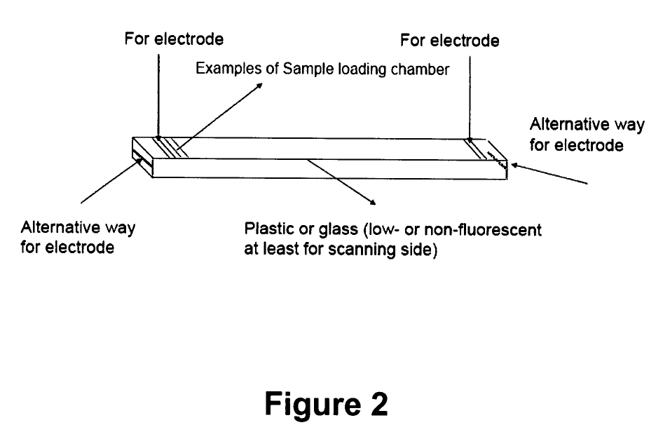

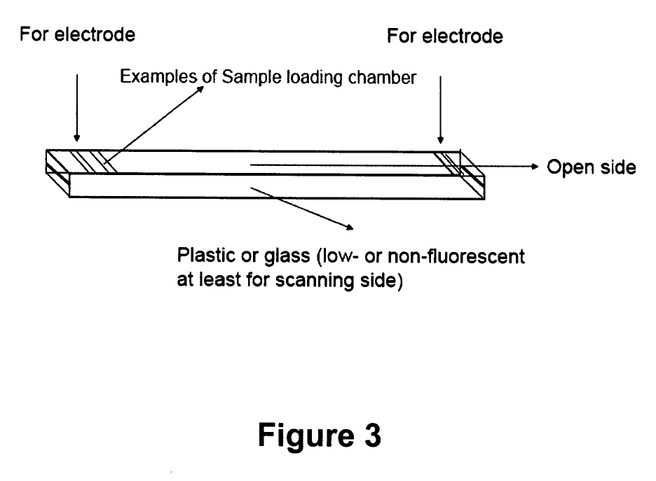

The channel of the isoelectric focusing chip can be sealed (FIG. 2) or open (FIG. 3). In particular embodiments, the channel of the isoelectric focusing chip is sealed along its length and additionally includes a sample port at the first end of the channel (FIG. 2). At least one side of the sealed chip (the “detection side”) must be sufficiently clear in the detection region to allow excitation light to enter the detection zone and emission light to reach an external detector. In addition, the material covering the detection region on the detection side of the chip is preferably a non-fluorescent or low-fluorescence material, as described above. In a variation of this embodiment, the channel is completely sealed, except for the openings for electrodes and the sample port, thus forming a chamber in which isoelectric focusing is carried out.

Isoelectric focusing can be carried out on chips according to the invention in any desired format. The invention includes chips adapted to perform the method described above in which isoelectric focusing is carried out in parallel pH gradients. Such chips can include a substrate having two or more parallel channels, with each channel having a receptacle for an electrode at each end and a detection region between the channel ends. Preferably, the substrate has a non-fluorescent or low-fluorescence material in the detection region of each channel. In a preferred embodiment, each channel is adjacent to at least one other channel, and the detection regions of each channel are aligned to permit detection by capture of a single image including all detection regions. As for single-channel chips, multi-channel chips can be sealed or unsealed. In particular multi-channel embodiments, the channels are sealed along their length, and each channel additionally comprises a sample port at one end of the channel. The sample port(s) can be, for example, a single opening extending over all channels or individual openings over each channel. In a variation of this embodiment, each channel is completely sealed, except for the openings for electrodes and the sample port, forming a plurality of chambers. In use, the same sample can added to one end of each channel and isoelectric focusing is carried out such that the same sample is separated in a different pH range in each channel.

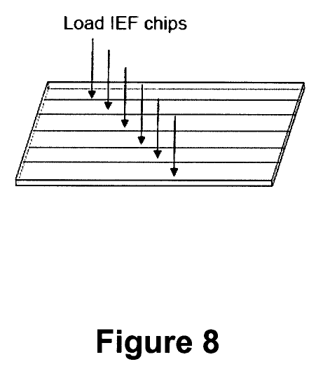

A multi-channel format can also be achieved by assembling individual isoelectric focusing chips (e.g., single-channel chips) in a chip holder that is adapted to hold a plurality of chips in parallel. See FIG. 8. The chip holder can be employed to hold the chip in position during isoelectric focusing and, preferably, during detection. To facilitate fluorescence detection, the chip holder can include a non-fluorescent or low-fluorescence material in the region that underlies the detection region of the chips positioned in the chip holder or can be made entirely of such material.

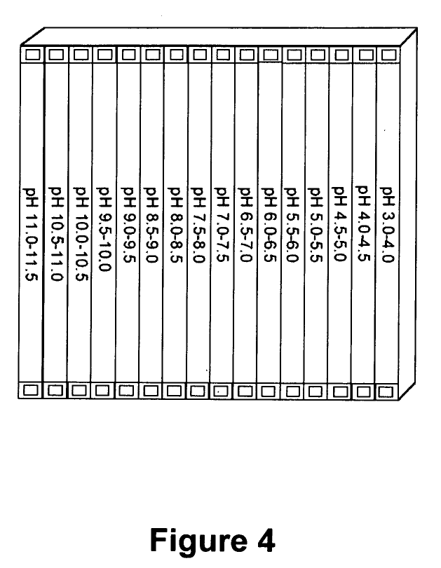

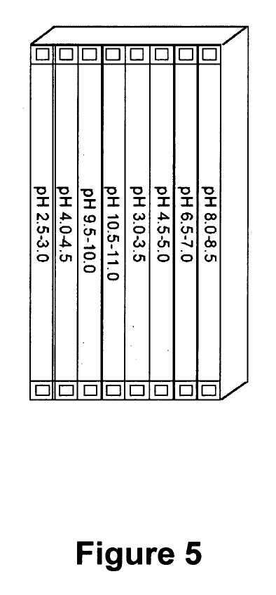

In particular embodiments, the isoelectric focusing chip includes a gel in each channel. The gel can be any gel suitable for carrying our isoelectric focusing, such as, polyacrylamide or agarose. The gel can include a pH gradient extending from the first end of the channel to the second end. In multi-channel embodiments, the pH gradient in each channel has a different pH range than that of the pH gradients of the other channels. The pH ranges, collectively, define a continuous pH range, as shown in FIG. 4, or a discontinuous pH range, as shown in FIG. 5.

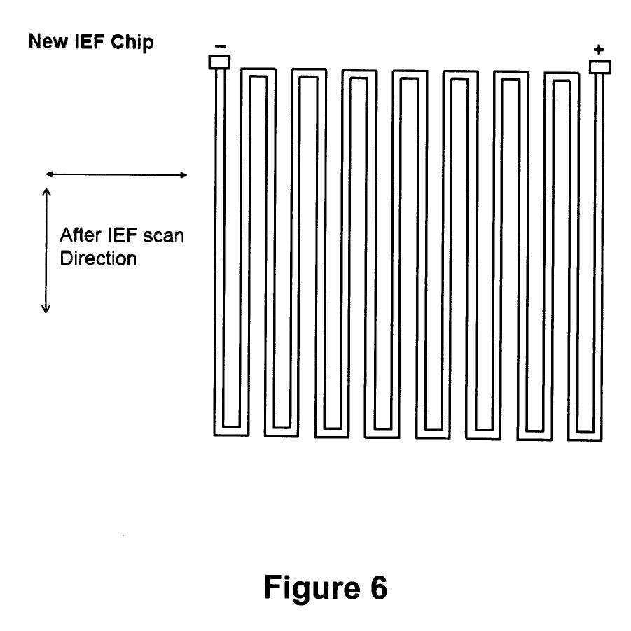

In another exemplary format, the isoelectric focusing chip can have a channel that defines a serpentine path in the substrate, as shown in FIG. 6. This format can be carried out using liquid or non-liquid isoelectric focusing. A continuous pH gradient is generated in the channel, such that a given long arm of the channel contains a different pH gradient that the adjacent long arms. This format is analogous to a multi-channel, continuous gradient format, as described above.

### III. Isoelectric Focusing Device

Another aspect of the invention is a device for running isoelectric focusing on one or more isoelectric focusing chips, such as those described above. The isoelectric focusing device includes a support capable of holding the isolectric focusing chip in position, and first and second electrodes for applying an electric field to an isoelectric focusing chip positioned on the support. The chip can be held in any suitable position, including a horizontal position or a vertical position. A voltage source is connected to the first and second electrodes. In particular embodiments, the support includes a non-fluorescent or low-fluorescence material in a region underlying the detection region of the isoelectric focusing chip. The support can be adapted to hold a chip holder, as described above which can, optionally, be removable to facilitate detection in a separate detector.

The isoelectric focusing device preferably included means for maintaining the temperature of the isoelectric focusing chip at a suitable temperature during isoelectric focusing. In general, the temperature is maintained between about 10° C. and about 20° C. In particular embodiments, temperature control is achieved using a Peltier element, which can, for example, be attached to, or integrated into the support. Peltier elements can be purchased from Ferrotec (USA) Corporation, Santa Clara, Calif.

In certain embodiments, the isoelectric focusing device can also include a fluorescence detector attached to the support at a position opposite the detection region of an isoelectric focusing chip positioned on the support. Any suitable fluorescence detector can be employed in the device. In particular embodiments, the detector is an electronic detection system such as a laser scanning system with a photo multiplier tube or a charged-coupled device (CCD) camera and a white light source.

The isoelectric focusing device can include sample delivery means for introducing sample into the first end(s) of the channel(s) of an isoelectric focusing chip positioned on the support. The sample delivery means can include a micropipette serviced by a pump or any other means known in the art of microsampling and automated analysis. The device can include multiple sample delivery means for introducing sample into multi-channel chips, for example.

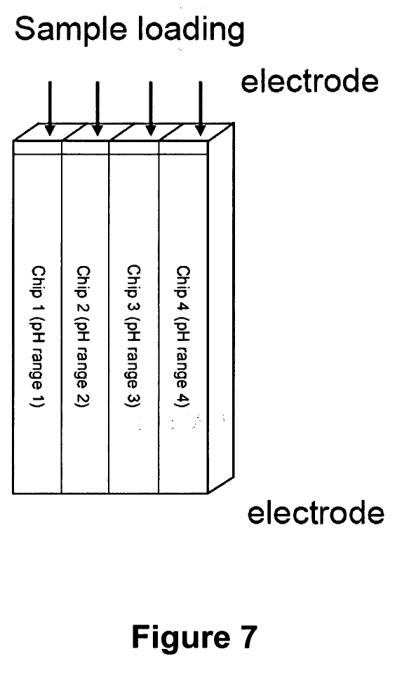

In particular embodiments, the isoelectric focusing device can be adapted to run isoelectric focusing on multiple isoelectric focusing chips simultaneously. Accordingly, the support can be designed to hold multiple chips in position, e.g., as shown in FIG. 7 and/or to hold a removable chip holder, e.g., as shown in FIG. 8. Depending on the configuration of the chips, the device can include an electrode that contacts all channels of the multiple chips at one end of the channels and another electrode that contacts all channels at the other end. Alternatively, the device can include an array of electrodes at each end of the channels. Similarly, the device can include an array of sampling means for simultaneously introducing sample into each chip.

Isoelectric focusing devices according to the invention can include an isoelectric focusing control unit attached to the support, wherein the isoelectric control unit controls flow of current to the first and second electrodes, or controls voltage and voltage-hours in linear or gradient mode.

In certain embodiments, the isoelectric focusing device includes a control unit linked to the sample delivery means and controls sample introduction. This sample delivery control unit can be separate from the isoelectric focusing control unit that controls flow of current to the first and second electrodes; but preferably the two functions are controlled by a single integrated unit.

The isoelectric focusing can include a control unit linked to the fluorescence detector that operated the detector. This detection control unit can be separate from the isoelectric focusing control unit (if present) and/or the sample delivery unit (if present). Preferably, however all functions are controlled by a single integrated unit.

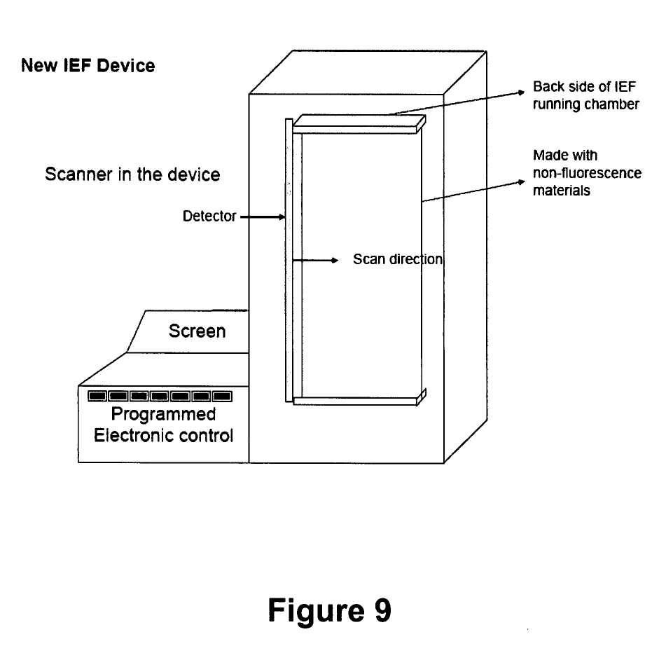

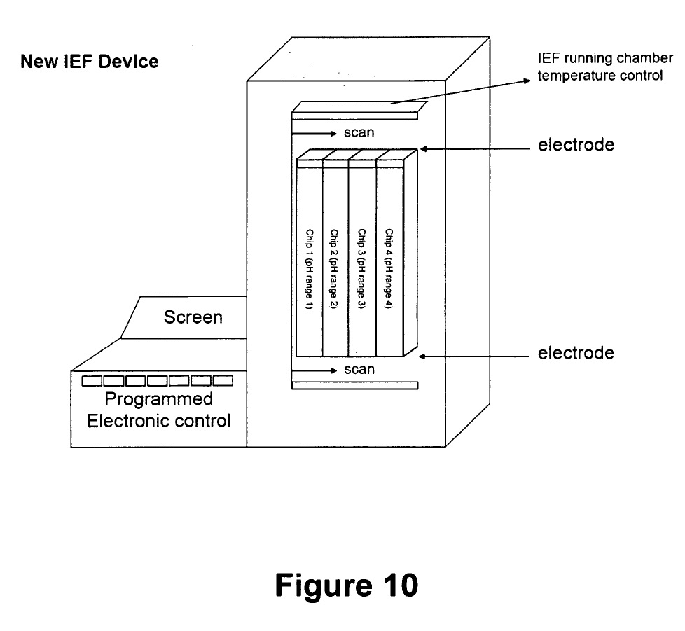

Any of the above-described control units can be manually operable and/or can include hardware and/or software elements to provide computer-controlled operations according to pre-defined or user-defined programs. An exemplary device with an integrated programmed electronic control is shown in FIG. 9 (empty) and FIG. 10 (holding multiple isoelectric focusing chips).

All publications cited herein are hereby expressly incorporated by reference.

This invention is further illustrated by the following specific, but non-limiting, examples. Procedures that are constructively reduced to practice are described in the present tense, and procedures that have been carried out in the laboratory are set forth in the past tense.

1. A method of comparing the protein composition of at least first and second samples, the method comprising:
(a) labeling proteins in the first sample with a first label, and labeling proteins in the second sample with a second, different label, wherein each label has ionic and pH characteristics whereby, upon isoelectric focusing, relative migration of a protein labeled with the first label is substantially the same as relative migration of the same protein labeled with the second label;
(b) subjecting the samples to isoelectric focusing in a first pH gradient having a first pH range to produce a pattern of separated proteins from both samples; and
(c) detecting the pattern of separated proteins prior to performing any additional separation step.

2. The method of claim 1, wherein no additional separation step is performed.

3. The method of claim 1, wherein said isoelectric focusing to produce a pattern of separated proteins from both samples is carried out simultaneously in one or more additional pH gradient(s) having a different pH range from the first pH range.

4. The method of claim 3, wherein said at least one additional pH gradient is adjacent, and parallel, to the first pH gradient, and the pattern of separated proteins in each gradient is detected by capturing a single image encompassing both patterns.

5. The method of claim 4, wherein isoelectric focusing is performed in a plurality of pH gradients in parallel, wherein each pH gradient has a different pH range from every other pH gradient.

6. The method of claim 5, wherein the pH ranges, collectively, define a continuous pH range.

7. The method of claim 5, wherein the pH ranges, collectively, define a discontinuous pH range.

8. The method of claim 1, wherein the at least first and second samples are mixed after labeling to form a sample mixture, and the sample mixture is applied to the first pH gradient.

9. The method of claim 1, wherein the comparison is quantitative.

10. The method of claim 1, wherein the samples are biological samples.

11. The method of claim 10, wherein the protein composition of a test sample is compared to the protein composition of a reference sample.

12. The method of claim 11, wherein the comparison provides information for diagnosis of one or more diseases or for monitoring the disease progression or treatment response.

13. The method of claim 1, wherein said isoelectric focusing is liquid isoelectric focusing.

14. The method of claim 1 wherein said isoelectric focusing is non-liquid isoelectric focusing.

15. The method of claim 1, wherein said isoelectric focusing is carried out on an isoelectric focusing chip.

16. The method of claim 15, wherein said isoelectric focusing to produce a pattern of separated proteins from both samples can carried out simultaneously in one or more additional pH gradient(s) having a different pH range from the first pH range.

17. The method of claim 16, wherein at least one additional pH gradient is adjacent, and parallel, to the first pH gradient, and the pattern of separated proteins in each gradient is detected by capturing a single image encompassing both patterns.

18. The method of claim 17, wherein isoelectric focusing is performed in a plurality of pH gradients in parallel, wherein each pH gradient has a different pH range from every other pH gradient.

19. The method of claim 18, wherein the pH ranges, collectively, define a continuous pH range.

20. The method of claim 18, wherein the pH ranges, collectively, define a discontinuous pH range.

21. The method of claim 15, wherein the isoelectric focusing chip is sealed.

22. The method of claim 1, wherein the proteins in the first and second samples are labeled to saturation.

23. The method of claim 1, wherein the first and second labels are luminescent labels, wherein each label emits luminescent light at a wavelength that is different from the wavelength at which luminescent light is emitted by any other label in the sample mixture.

24. The method of claim 23, wherein the first and second labels are fluorescent labels.

25. The method of claim 24, wherein said isoelectric focusing is carried out on an isoelectric focusing chip, wherein at least one surface of said chip comprises a non-fluorescent or low-fluorescence material.

26. An isoelectric focusing chip comprising:
a substrate comprising a channel for carrying out isolectric focusing, the channel comprising first and second ends, and a detection region between the first and second ends;
wherein each end of the channel comprises a receptacle for an electrode;
wherein the substrate comprises a non-fluorescent or low-fluorescence material in the detection region of the channel.

27-37. (canceled)

38. An isoelectric focusing device comprising:
a support capable of holding an isolectric focusing chip in position, wherein the support comprises a non-fluorescent or low-fluorescence material in a region underlying the detection region of the isoelectric focusing chip;
first and second electrodes for applying an electric field to an isoelectric focusing chip positioned on the support; and
a voltage source connected to the first and second electrodes.

39-46. (canceled)
